(19) United States
(12) Patent Application Publication
(10) Pub. No.: US 2022/0152093 A1
(43) Pub. Date: May 19, 2022

(54) OPTIMIZED NUTRIENT SALT COMPOSITION

(71) Applicant: JOLLY MATHEW VADAKKEMURI, ERNAKULAM (IN)

(72) Inventor: JOLLY MATHEW VADAKKEMURI, ERNAKULAM (IN)

(21) Appl. No.: 16/602,194

(22) PCT Filed: Jan. 31, 2018
(86) PCT No.: PCT/IN2018/000009

§ 371 Date: Aug. 22, 2019

(51) Int. Cl. A61K 33/00; A61K 47/02; A61K 31/522; A61K 36/74; A61K 36/82; A61K 36/185; A61K 31/14; A61K 31/685; A61K 9/00; A23L 27/40; A23L 2/60; A23L 33/16; A23L 33/125; A23B 7/157; A23B 4/027; A23L 33/15

(52) U.S. Cl. CPC A61K 33/00; A23V 2002/00; A61K 31/522; A61K 36/74; A61K 36/82; A61K 36/185; A61K 31/14; A61K 31/685; A61K 9/0053; A23L 27/40; A23L 2/60; A23L 33/16; A23L 33/125; A23B 7/157; A23B 4/027; A23L 33/15; A61K 47/02

(57) ABSTRACT

The invention relates to an optimized nutrient salt composition comprising elemental sodium and potassium in the ratio of 1:1.5 to 1:7 preferably 1:3 and elemental potassium to chloride in the ratio of 1:1 to 1:1.5 and method of preparing the same. Said composition can be optionally added with a) salts of calcium and, or magnesium as taste stabilizers, b) carbohydrate like polysaccharide, oligosaccharide, disaccharide, monosaccharide to produce energy electrolyte powder, c) food additives selected from anti-caking agents, crystal modifying agents, flavoring agents, flavor modifying agents, coloring agents and or any other food additives and d) nutrients selected from proteins, amino acids, carbohydrates, sugars, fat/oil, fatty acids, vitamins, minerals and water. Said optimized nutrient salt composition can be used as food, food ingredient, food supplement, table salt, preservative, food additive, seasoning agent and ingredient for oral rehydration fluid, intravenous fluids, beverages, energy drinks, sports/study drinks and stimulant drinks for humans, pets and livestock and for plant nutrition.

### FIELD OF THE INVENTION AND USE OF INVENTION

This invention relates to an optimized nutrient salt composition having salts of sodium and potassium wherein the ratio of elemental sodium to elemental potassium is maintained at a ratio ranging from 1:1.5 to 1:7 preferably 1:3 and elemental potassium to chloride is maintained at a ratio ranging from 1:1 to 1:1.5. It is capable of using as a food, a food ingredient, a food supplement, table salt, a preservative, a food additive, a seasoning agent, an ingredient for oral rehydration fluid, an ingredient for pharmaceutical preparation of intravenous fluids, an ingredient of beverages, energy drinks, sports/study drinks and stimulant drinks for humans, pets and livestock and for plant nutrition.

### PRIOR ART AND PROBLEM TO BE SOLVED

Sodium and potassium are the major positively charged ions and chloride is the major negatively charged ion in the body. Conduction of electrical nerve impulses depends on active transport of sodium and potassium ions across the cell membrane with the help of sodium potassium pump. The movement of positively charged sodium and potassium is always accompanied by negatively charged ions mainly chloride. Chloride ions have other important functions in digestive system including production of hydrochloric acid of gastric juice in the stomach and assisting absorption of vitamins, mineral and other nutrients. Chloride plays important role in acid base balance and transport of carbon dioxide and its exchange between erythrocyte cell membrane and interstitial fluid.

Evolutionally as a terrestrial animal human kidney has the capacity to retain sodium and can live with as low as around 500 mg of sodium per day. But our capacity to retain potassium and chloride is low. The major dietary source of sodium is common salt. Pulses, animal products, cereals, fruits and vegetables are good sources of potassium. Only few food items like sea weeds, olive, celery, lettuce and tomato contain substantial quantity of chloride. Most of our chloride requirement is met from the sodium chloride added during cooking.

Industrialization, mass cultivation, processing of food materials, preservation of food and food additives like flavor enhancing agents, texture modifying agents, food colors etc. have made significant changes in the composition of food we eat. During processing of food there is significant loss of essential nutrients like vitamins, minerals, essential fat and protein. Different sodium salts like sodium chloride, sodium citrate, monosodium gluconate, sodium benzoate, sodium bicarbonate etc. are added during processing as food additives. This increases the daily intake of sodium in the food which has deleterious effects on our health and is closely related to the pathogenesis of life style diseases like hypertension, cardiovascular diseases, stroke, urinary stone and osteoporosis.

Various efforts have been made to develop low sodium salt aiming to reduce sodium intake without compromising the taste of food during the last century. There are many patents and patent applications on this ground including U.S. Pat. Nos. 9,149,061 B2; 4,322,407 A; CN 102423033 A; EP 0766927 A1; US 20040224076 A1; EP 0291578 A1; US 20070059428 A1; U.S. Pat. No. 4,473,595 A; US 20110229607 A1 and U.S. Pat. No. 6,896,925 B2. All these patents are aiming at giving salty taste to food with low sodium content.

Even with all this effort the daily intake of sodium in our food is high and the latest recommendations in Dietary guidelines for Americans 2015-2020 emphasizes on reducing the intake of sodium. As per recommended dietary allowance sodium intake should be kept below 2300 mg/day for a healthy adult and DASH diet (dietary approach to stop hypertension) encourages to reduce the sodium in the diet below 1500 mg to help lower blood pressure. On average, American adults eat more than 3,400 milligrams (mg) of sodium each day, which is significantly higher than the recommended limit.

Many studies show modern food is deficient in potassium. During the processing of food materials a good amount of potassium in the food is lost. US Food and Nutrition Board recommends adults over the age of 19, adolescents between 14 and 18 years old and pregnant women should consume 4,700 milligrams of potassium each day. Nursing women need more around 5,100 milligrams daily. A dietary survey indicated that the average dietary potassium intake is about 2,300 mg/day for adult women and 3,100 mg/day for adult men which are low.

### OBJECTIVES OF THE INVENTION

The main objective of the invention is to provide an optimized nutrient salt composition containing sodium, potassium and chloride in optimal proportion and capable of using as food, food ingredient, food supplement, table salt, preservative, food additive, seasoning agent, ingredient for oral rehydration fluid, ingredient for pharmaceutical preparation of intravenous fluids, ingredient for beverages, energy drinks, sports/study drinks and stimulant drinks for humans, pets and livestock and for plant nutrition.

### SUMMARY OF THE INVENTION

The invention discloses an optimized nutrient salt composition capable of using as a food, a food ingredient, a food supplement, table salt, a preservative, a food additive, a seasoning agent, an ingredient for oral rehydration fluid, an ingredient for pharmaceutical preparation of intravenous fluids, an ingredient of beverages, energy drinks, sports/study drinks and stimulant drinks for humans, pets and livestock and for plant nutrition. It is comprising of elemental sodium and potassium in the ratio ranging from 1:1.5 to 1:7 preferably 1:3 and elemental potassium and chloride in the ratio ranging from 1:1 to 1:1.5 preferably 1:1.4 in the composition:

The source for sodium is selected from sodium chloride, sodium glutamate, sodium bicarbonate, sodium gluconate, sodium citrate, sodium phosphate, sodium ascorbate, sodium iodide and any other edible sodium salts. The source for potassium is selected from potassium chloride, potassium phosphate, potassium bicarbonate, potassium gluconate, potassium aspartate, potassium orotate, potassium glutamate, potassium citrate, potassium iodide and any other edible potassium salts. The source for chloride is selected from sodium chloride, potassium chloride, calcium chloride, magnesium chloride, zinc chloride, choline chloride and any other edible chloride salts.

In another embodiment of the invention the optimized nutrient salt composition is enriched with essential or non-essential nutrients, vitamins, minerals, amino acids, proteins, fats/oils, fatty acids and carbohydrates for human food, pets and animal food and plant nutrition. In another embodiment the optimized nutrient salt composition may be added with one or more food additives selected from, anti-caking agents, crystal modifying agents, flavoring agents, flavor modifying agents, coloring agents and, or any other food additives.

In yet another embodiment of the invention, the optimized nutrient salt composition is added with salts of calcium and, or magnesium as taste stabilizer. Edible salts of calcium and magnesium including calcium carbonate, magnesium carbonate, magnesium oxide, calcium phosphate, magnesium phosphate, calcium citrate and other edible calcium and magnesium salts are selected as taste stabilizer. In another embodiment of the invention calcium carbonate is added as taste stabilizer.

According to another embodiment under the invention, in the optimized nutrient salt composition, sodium chloride is selected as source for sodium and chloride and potassium chloride is selected as source for potassium and chloride and a ratio of sodium chloride and potassium chloride ranges from 1:1.2 to 1.25:7 preferably 3:7. In a preferred embodiment under the invention, stabilized optimized nutrient salt is enriched with carbohydrate including starch, polysaccharide, oligosaccharide, disaccharide and, or monosaccharide to produce energy electrolyte powder which can be reconstituted with water at convenience to make energy electrolyte solution/oral rehydration fluid.

The method of preparation of the optimized nutrient salt composition comprising, selecting the source of salts from edible salts of food grade/pharmaceutical grade salts of sodium, salts of potassium and other chloride salts; calculating the amount of chloride, sodium and potassium in the selected salts as per standard chemistry practice considering the chemical formula of the salt; finding out the proportion of each salt to be taken to maintain the ratio of sodium to potassium in the salt composition in the range of 1:1.5 to 1:7 and potassium to chloride in the range of 1:1 to 1:1.5 using standard chemistry calculation methods; measuring the selected salts as per calculation; powdering the salts in a pulverizer; combining the salt composition in a mixing chamber at 200-500 RPM for 10 to 20 minutes to get an even mixture; optionally adding, calcium/magnesium salt as taste stabilizers; nutrients like carbohydrates, fats/oils, fatty acids, proteins, amino acids, vitamins and minerals; and food additives including anti caking agents, crystal modifying agents, flavoring agents or any other food additives; and packing the product in to air tight containers to avoid moisture ingress.

The method of preparation of electrolyte energy drink using stabilized optimized nutrient salt includes enrichment with carbohydrate including starch/polysaccharides, oligosaccharide, disaccharide and, or monosaccharide to produce energy electrolyte powder which can be reconstituted with water at convenience to make energy electrolyte solution/oral rehydration fluid.

The method of preparation of sports/study drink using stabilized optimized nutrient salt include enrichment with carbohydrate selected from starch, oligosaccharide, sugar, glucose, fructose and any other carbohydrate and choline selected from edible choline source including choline bitartarate, choline chloride, lecithin, phosphatidylcholine or any other edible choline to produce sports/study drink powder which is dissolved in water to make sports/study drink solution.

The method of preparation of stimulant drink using optimized nutrient salt include enrichment with carbohydrate selected from polysaccharide starch, glycogen or any other polysaccharide; oligosaccharide, disaccharide including sucrose, lactose, or any other disaccharide and monosaccharide including glucose, fructose or any other monosaccharide; and choline selected from edible choline source including choline bitartarate, choline chloride, lecithin, phosphatidylcholine or any other edible choline; and stimulant drugs is added which is selected from caffeine, or any other stimulant drugs, coffee extract, tea extract, coco extract or any other stimulant natural extract to produce stimulant drink powder which is dissolved in water to make stimulant drink solution.

In the method of preparation of intra venous electrolyte solution, optimized nutrient salt composition is subjected to an additional step of filtration and ultra-purification and is mixed with ultra-purified water to make intra venous fluid as per known pharmaceutical technologies. The resultant product is capable of using as intravenous fluid for supporting fluid and electrolyte balance. The method of production of intravenous nutrition includes enrichment of the said intravenous fluid with one or more ultra-purified nutrients selected from glucose, vitamins, minerals, amino acids, triglycerides, fatty acids and any other nutrients as per known pharmaceutical technologies which is capable of using as intra venous nutrition.

The constituents of salts in this optimized nutrient salt composition and, or its solution when taken as food acts as means for stabilization of sodium potassium pump at cellular level in multicellular organisms, provide electrolytes in calorie restricted diet of obese patients, compensate electrolyte loss through sweat, stool, plasma, serum or urine. It can also function as preservative for food.

### BRIEF DESCRIPTION OF THE DRAWING

An exemplary embodiment of the present invention is illustrated by way of example in the accompanying drawings and the invention will become better understood when the following detailed description is read with reference to the accompanying drawings.

FIG. 1 shows a flow diagram depicting the hypothesis leading to the special sodium potassium ratio 1:1.5 to 1:7 preferably 1:3 in optimized nutrient salt composition.

FIG. 2 shows a flow diagram depicting the method of production of optimized nutrient salt with elemental sodium to potassium ratio 1:1.5 to 1:7 preferably 1:3 and elemental potassium to chloride ratio 1:1 to 1:1.5.

FIG. 3 shows a flow diagram depicting the method of taste stabilization of optimized nutrient salt using calcium and, or magnesium salt.

FIG. 4 shows a flow diagram depicting the method of production of enriched optimized nutrient salt using sodium chloride, potassium chloride, nutrients and food additives.

FIG. 5 shows a flow diagram depicting the method of production of optimized nutrient salt using sodium chloride and potassium chloride and taste stabilization of the optimized nutrient salt with calcium carbonate.

FIG. 6 shows a flow diagram depicting the method of production of optimized nutrient salt using sodium glutamate and potassium chloride and taste stabilization with magnesium carbonate.

FIG. 7 shows a flow diagram depicting the method of production of optimized nutrient salt using sodium bicarbonate, potassium gluconate and calcium chloride.

FIG. 8 shows a flow diagram depicting the method of production of electrolyte energy powder and electrolyte energy solution using optimized nutrient salt.

FIG. 9 shows a flow diagram depicting the method of production of sports/study drink powder and sports/study drink solution using optimized nutrient salt.

FIG. 10 shows a flow diagram depicting the method of production of stimulant sports/study drink powder and stimulant sports/study drink solution using optimized nutrient salt.

FIG. 11 shows a flow diagram depicting the method of production of intravenous fluid using optimized nutrient salt.

FIG. 12 shows a flow diagram depicting the method of production of intravenous nutrition using optimized nutrient salt.

### DETAILED DESCRIPTION OF THE INVENTION

The invention is described in detail in the following description. The present invention provides an optimized nutrient salt composition having elemental sodium and potassium in the ratio ranging from 1:1.5 to 1:7 while ratio of potassium to chloride is maintained at a ratio in the range of 1:1 to 1:1.5.

This has multifarious use such as food, food ingredient, food supplement, table salt, preservative, food additive, seasoning agent, ingredient for oral rehydration fluid, ingredient for pharmaceutical preparation of intravenous fluids, ingredient of beverages, energy drinks, sports/study drinks and stimulant drinks for humans, pets and livestock and for plant nutrition. It can also be enriched with essential or non-essential nutrients for human food, pets and animal food and plant nutrition. Said composition can also be added with anti-caking agents and, or crystal modifying agents and, or flavoring agents and, or nutrients including proteins, amino acids, carbohydrates, sugars, fats/oils, fatty acids, vitamins, minerals, water and, or any other nutrients or food additives.

A method of preparation of said optimized nutrient salt composition also is disclosed. It comprise of selecting the source of salts from edible salts of food grade/pharmaceutical grade salts of sodium, salts of potassium and other chloride salts; calculating the amount of chloride, sodium and potassium in the selected salts as per standard chemistry practice considering the chemical formula of the salt; finding out the proportion of each salt to be taken to maintain the ratio of sodium to potassium in the salt composition in the range of 1:1.5 to 1:7 and potassium to chloride in the range of 1:1 to 1:1.5 using standard chemistry calculation methods; measuring the selected salts as per calculation; powdering the salts in a pulverizer; combining the salt composition in a mixing chamber at 200-500 RPM for 10 to 20 minutes to get an even mixture; optionally adding calcium and, or magnesium salt as taste stabilizer; nutrients including vitamins, minerals, amino acids, proteins, fats/oil, fatty acids, carbohydrates or any other nutrients; and food additives including anti caking agents, crystal modifying agents, flavoring agents or any other food additives; and packing the product in to air tight containers to avoid moisture ingress.

The optimized nutrient salt composition can be subjected to filtration and ultra-purification and can be mixed with water to make intra venous fluid. The resultant intravenous fluid with optimized salt composition can be used for supporting fluid and electrolyte balance. In addition the resultant intravenous fluid with optimized nutrient salt composition can be added with other nutrients to provide energy and nutrition for human, pets and livestock.

The synergetic effect of the composition was evident from the result we observed when used as detailed in examples, which are appended in the succeeding paragraphs. The reasoning for the specific results with the use of optimized nutrient salt at various circumstances can be attributed to special ratio of essential minerals sodium, potassium and chloride in the composition.

Health issues like hypertension, cardiovascular disease and stroke are not only related to high sodium content but also to low potassium content of food.

During the processing of food materials a good amount of potassium in the food is lost. Low sodium salt and low sodium diet can lead to reduction in chloride intake which can lead to fatigue and related health problems as there are only a few food items that contain substantial amount of chloride.

Continuous supply of major electrolytes sodium, potassium and chloride is essential for optimal cell and body function as these elements are not stored in the body like other electrolytes calcium or magnesium. As only few farm produces give substantial amount of chloride in the diet, in the current life style body depends on common salt as the major source for chloride. Effort to reduce sodium by reducing common salt in diet can lead to chloride deficiency leading to tiredness, fatigue and low productivity.

Potassium is the principal positively charged ion in the intracellular fluid while sodium is the principal positively charged ion in the extracellular fluid. The concentration differences between potassium and sodium across cell membranes create an electrochemical gradient known as the membrane potential. A cell's membrane potential is maintained by ion pumps in the cell membrane, the sodium-potassium-ATPase pumps. These pumps use ATP as energy to pump sodium out of the cell in exchange for potassium against a concentration gradient. The static membrane potential of quiescent cells is called the resting membrane potential. In excitable tissues like neurons, muscles, and some secretory cells in glands action potential is generated with stimulation. Membrane voltage in the majority of non-excitable cells can also undergo changes in response to environmental or intracellular stimuli. Membrane voltage changes like action potential are caused by specific changes in membrane permeability for potassium, sodium, chloride and calcium ions, which in turn result from changes in activity of various ion channels, ion transporters, and exchangers. The activity of sodium-potassium pump is by which three cations of sodium are pumped out of the cell and two cations of potassium are pumped in to the cell. The positively charged ions sodium and potassium are always accompanied by negatively charged chloride or any other negatively charged ions like bicarbonate, phosphate, sulphate or protein.

The sodium potassium pump activity has been estimated to account for 20%-40% of the resting energy expenditure in a typical adult. During intellectual and physical activity the energy expenditure for sodium potassium ATPase activity will be very high due to nerve impulse transmission and muscle contraction. The large proportion of energy dedicated to maintaining sodium/potassium concentration gradients emphasizes the importance of this function in sustaining life. Tight control of cell membrane potential is critical for nerve impulse transmission, muscle contraction, and heart function. A negatively charged ion mostly chloride ion accompany the movement of sodium or potassium across the cell membrane.

Potassium concentration inside the cell is around 150 meq/l and outside the cell is around 5 meq/l. Sodium concentration inside the cell is around 14 meq/l and outside the cell is around 140 meq/l. Potassium concentration is about 30 times higher inside the cells than outside the cells, while sodium concentrations are ten times lower inside the cells than outside the cells. The gradient of sodium and potassium concentration across the cell is around 1:3.

In humans 0.2% of total body mass is sodium and 0.4% of total body mass is potassium and the sodium to potassium ratio in the body is 1:2. In the fluid compartment of the body total quantity of sodium ions and potassium ions ratio is around 1:2.5.

As per the standard nutritional recommendation sodium intake is to be restricted below 2300 mg per day and potassium intake per day should be 3600 mg per day, where the ratio of sodium to potassium is 1:1.5. As per the DASH diet sodium intake per day is to be reduced below 1500 mg and potassium intake above 4500 mg, where the ratio of sodium to potassium is 1:3.

Evolutionally humans as a terrestrial animal considering the low availability of sodium in the land is physiologically equipped to conserve sodium and can live with as minimum as 500 mg sodium per day. But human kidney and physiology are not equipped to conserve potassium as the availability of potassium in the land is plenty. In Paleolithic diet potassium content per day was above 7000 mg/day and sodium content was below 1000 mg especially in interior high lands, where the sodium potassium ratio was around 1:7 or above.

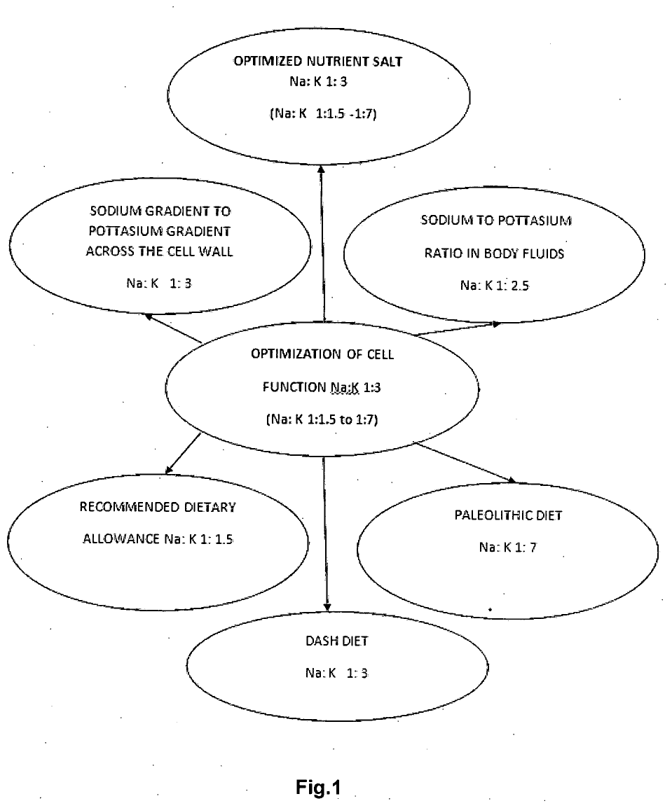

In an embodiment of the invention as depicted in FIG. 1 shows the hypothesis leading to the special sodium potassium ratio 1:3 in optimized nutrient salt composition. Considering the distribution of sodium and potassium in the intra cellular and extracellular fluid, sodium gradient to potassium gradient across the cell membrane, energy needed for sodium potassium pump activity, amount of sodium ions and potassium ions in the body's fluid compartment and human physiology regarding sodium and potassium conservation and excretion, it is hypothesized that “maintaining the ratio of sodium and potassium in the diet around 1:1.5 to 1:7 preferably around 1:3 is essential for optimization of cell function, body functions and health”. Maintenance of sodium and potassium concentration across the cell membrane will be less stress full for the cells and the body if the ratio of sodium and potassium in the diet is kept around 1:3. For maintaining optimal health, in moderate climatic conditions and physical activity, dietary intake of sodium should be around 1200 mg and potassium should be around 3600 mg/day, where the ratio of sodium and potassium is 1:3.

Recommended dietary requirement for chloride varies with physical activity and climatic conditions. Recommended Daily Value is 3400 mg/day. Chloride ion moves with sodium and potassium ions in nerve conduction. Chloride loss occurs in sweat, urine, and stool. During vomiting and diarrhoea, chloride loss is out of proportion to sodium loss because gastric secretion contains hydrochloric acid and its main constituent by weight is chloride. Chloride loss occurs in blood loss and burns through plasma and serum respectively. Hypochloraemia leads to alkalosis with acid base imbalance which is a life threatening condition. Best source for chloride replacement is sodium chloride as it contains 60% chloride and the accompanying sodium also is needed substantially. In many medical situations treatment with intra venous normal saline and oral rehydration fluid the recovery is mainly related to chloride replacement rather than sodium. Chloride is easily excreted through stool, urine and sweat. Undesirable side effect associated with sodium chloride is related to accumulation of sodium and accumulation of chloride does not occur if the water consumption is sufficient.

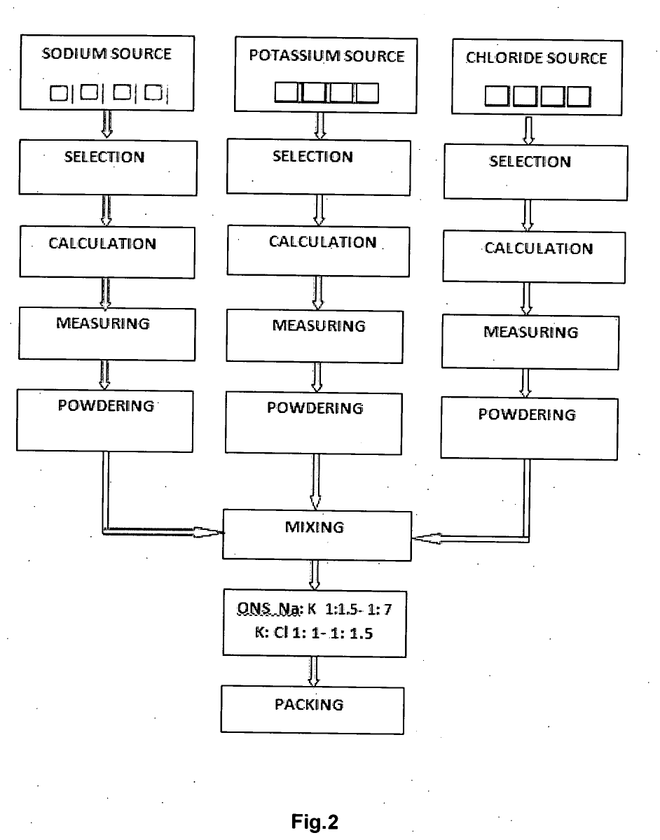

In another embodiment of the invention as depicted in flow diagram FIG. 2 shows the method of production of optimized nutrient salt maintaining elemental sodium and potassium in the ratio ranging from 1:1.5 to 1:7 preferably 1:3 and elemental potassium to chloride in the ratio ranging from 1:1 to 1:1.5 using edible chloride, sodium and potassium salt.

For production of optimized nutrient salt composition, suitable edible chloride, sodium and potassium salts are selected considering the expected nutritional aspects, flavour and physical and chemical characteristics of the salts. Source for chloride includes sodium chloride, potassium chloride, calcium chloride, magnesium chloride, zinc chloride, choline chloride and other edible chloride salts. Source for sodium include sodium chloride, sodium glutamate, sodium bicarbonate, sodium gluconate, sodium citrate, sodium phosphate, sodium ascorbate, sodium iodide and any other edible sodium salts. Source for potassium include potassium chloride, potassium phosphate, potassium bicarbonate, potassium gluconate, potassium glutamate, potassium, aspartate, potassium orotate, potassium citrate, potassium iodide and other edible potassium salts.

The steps involved in production of optimized nutrient salt composition include; a) selecting the source of salts from edible salts of food grade/pharmaceutical grade salts of sodium, salts of potassium and chloride salts; b) calculating the amount of chloride, sodium and potassium in the selected salts as per standard chemistry calculation methods considering the chemical formula of the salt; c) finding out the proportion of each salt to be taken to maintain the ratio of sodium to potassium in the salt composition in the range of 1:1.5 to 1:7 using standard chemistry calculation methods and potassium to chloride in the range of 1:1 to 1:1.5 using standard chemistry calculation methods; d) measuring the selected salts as per calculation; e) powdering the salts in a pulverizer; f) combining the salt composition in a mixing chamber at 200-500 RPM for 10 to 20 minutes to get an even composition; g) taste stabilization by adding calcium and, or magnesium salt in to the combination; h) mixing the composition in a mixing chamber at 200-500 RPM for 10 to 20 minutes to get an even composition; i) enriching with nutrients and food additives including anti caking agents, crystal modifying agents, flavoring agents or any other food additives; and j) packing the product of step i) in to air tight containers to avoid moisture ingress.

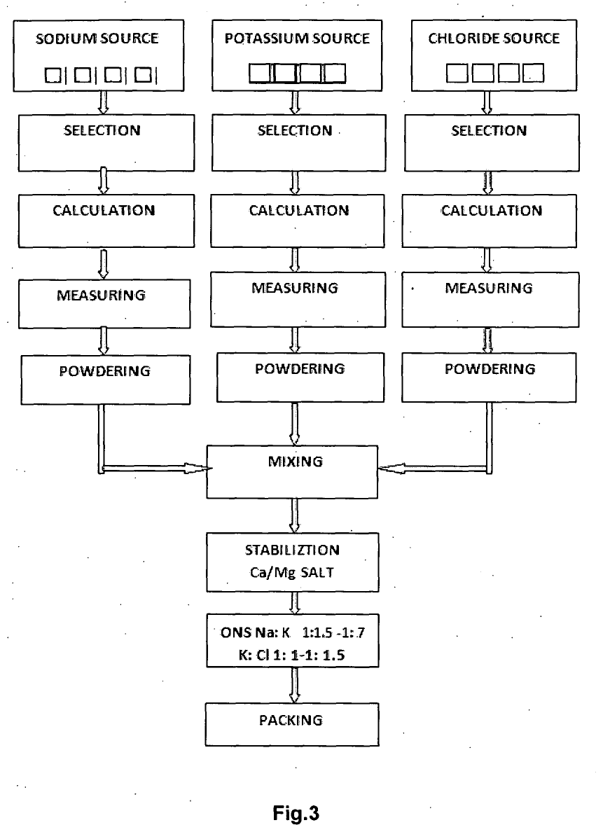

In another embodiment of the invention as depicted in flow diagram FIG. 3 shows the method of stabilization of taste of optimized nutrient salt. A major problem encountered with potassium salt is the metallic taste and bitter after taste when used as food ingredient. This problem can be solved by adding a taste stabilizer in the composition. Edible salts of calcium and magnesium including calcium carbonate, magnesium carbonate, magnesium oxide, calcium phosphate, magnesium phosphate, calcium citrate, calcium chloride, magnesium chloride and any other edible calcium and, or magnesium salts and a combination thereof can be used as taste stabilizers.

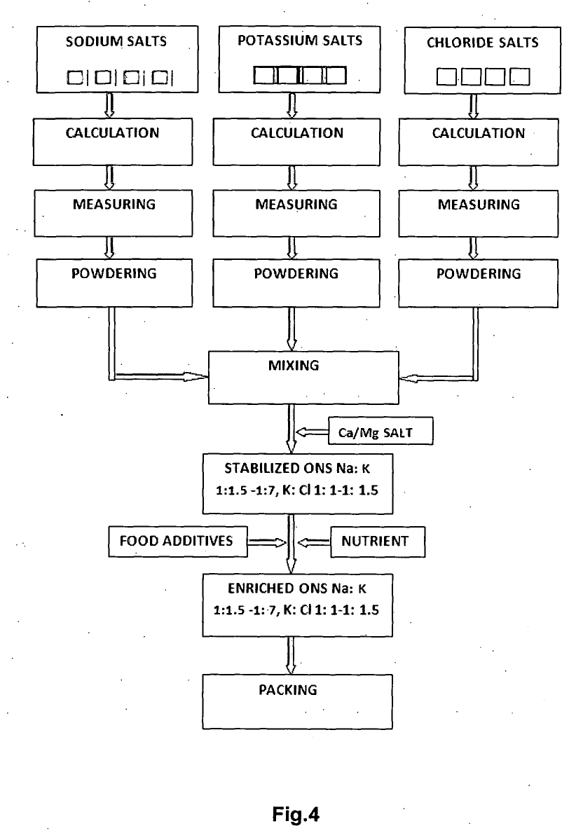

In another embodiment of the invention as depicted in flow diagram FIG. 4 shows the method of production of enriched optimized nutrient salt by adding nutrients and food additives to optimized nutrient salt. Nutrients including vitamins, minerals, fats/oils, fatty acids, carbohydrates, proteins, amino acids and food additives including flavouring agents, flavour modulators, food colours, anticaking agents and any other food additives or nutritionally important materials can be added for making nutrient fortified salt, oral rehydration fluid, soft drink, sports drink, energy dink and other food items and food supplements. Adding drugs like caffeine and other stimulant drugs, coffee extract, tea extract, coco extract and, or other stimulant herbal extracts can make stimulant drinks with optimal sodium, potassium and chloride ratio.

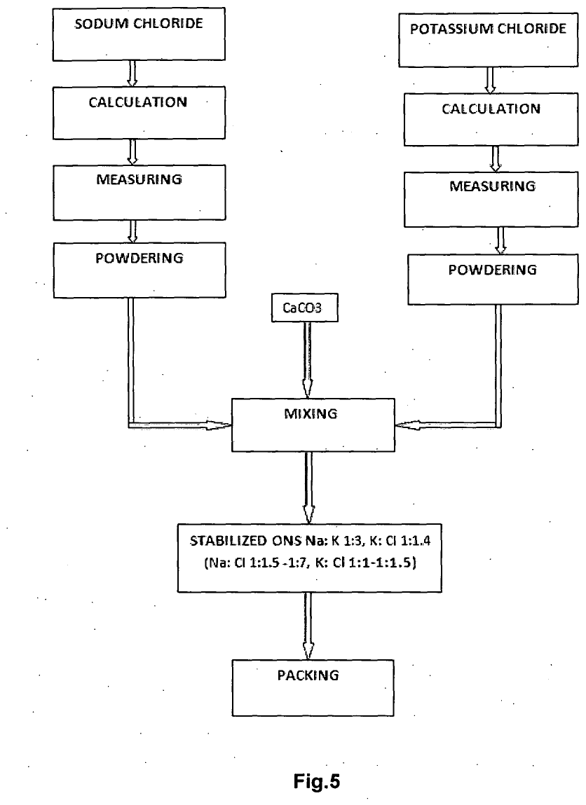

In another embodiment of the invention as depicted in flow diagram FIG. 5 shows the method of production of optimized nutrient salt with sodium to potassium in the ratio of 1:1.5 to 1:7 and potassium to chloride in the ratio of 1:1 to 1:1.5 using sodium chloride as source for sodium and chloride and potassium chloride as source for potassium and chloride. Stabilization of salt composition is done with edible salts of calcium and or magnesium 0.5% to 5% of the total weight. Calcium and magnesium salt give added nutritional benefit as they are essential elements and act as electrolytes, important for cellular functions while dissolved in water.

Considering the molecular weight of sodium chloride and potassium chloride, percentage of elemental sodium in sodium chloride and elemental potassium in potassium chloride is calculated. Sodium chloride contains 39.34% sodium and 60.66% chloride, potassium chloride contains 52.45% potassium and 47.55% chloride. Proportion of sodium chloride and potassium chloride needed for the composition depends on the ratio of sodium and potassium needed in the optimized nutrient salt. For getting sodium and potassium in the ratio of 1:1.5 to 1:7, sodium chloride and potassium chloride ratio ranges from 1:1.2 to 1.25:7 respectively.

In an embodiment of method of production of optimized nutrient salt with sodium to potassium in the ratio of 1:3 and potassium, to chloride in the ratio of 1:1.4, where sodium chloride is selected as the source for sodium and chloride and potassium chloride is selected as source of potassium and chloride. Calculation is done considering the molecular weight of sodium chloride and potassium chloride, percentage of elemental sodium and chloride in sodium chloride and elemental potassium and chloride in potassium chloride as in standard chemistry practice. Molecular weight of sodium chloride is 58.44 g/mole and on calculation considering molecular weight of sodium and chloride it contains 39.34% sodium and 60.66% chloride. Molecular weight of potassium chloride is 74.55 and as per standard chemistry calculation, it contains 52.45% potassium and 47.55% chloride. Considering these factors, as per standard method of chemistry calculations, to get the optimized nutrient salt of sodium to potassium in the ratio of 1:3 and potassium to chloride in the ratio of 1:1.4, sodium chloride and potassium chloride has to be taken in the ratio 3:7. In this embodiment calcium carbonate is selected as taste stabilizer. Salts are measured as 30% sodium chloride and 70% potassium chloride. The salts are powdered individually and mixed in a mixing chamber. Stabilization is done by adding calcium carbonate 1% of the total weight of the composition. 10 gm of this optimized nutrient salt prepared as per this embodiment give around 3600 mg potassium and 1200 mg sodium and 5200 mg chloride which satisfy recommended dietary allowance for a day. Chloride content in this 10 gm is around 5200 mg which is higher than the recommended dietary intake. Excess chloride is easily excreted through bowel, kidney and skin if the water intake is sufficient and no toxicity occurs. High chloride content of this embodiment is beneficial when optimized nutrient salt is used as preservative. In this embodiment of optimized nutrient salt saltiness of the composition is almost half compared to common salt, with same saltiness we can get the benefit of more potassium and chloride. Chloride content can be further increased by adding calcium chloride which contains around 79% chloride with added benefit of essential nutrient calcium 21%. Magnesium chloride and choline chloride can also be added to increase the chloride content along with additional nutritional benefit of essential nutrient magnesium and choline.

In another embodiment of the invention where ratio of potassium to chloride is 1:1, part of potassium chloride is replaced by other potassium salts including potassium phosphate, potassium sulphate, potassium gluconate, potassium iodide or any other edible potassium salt.

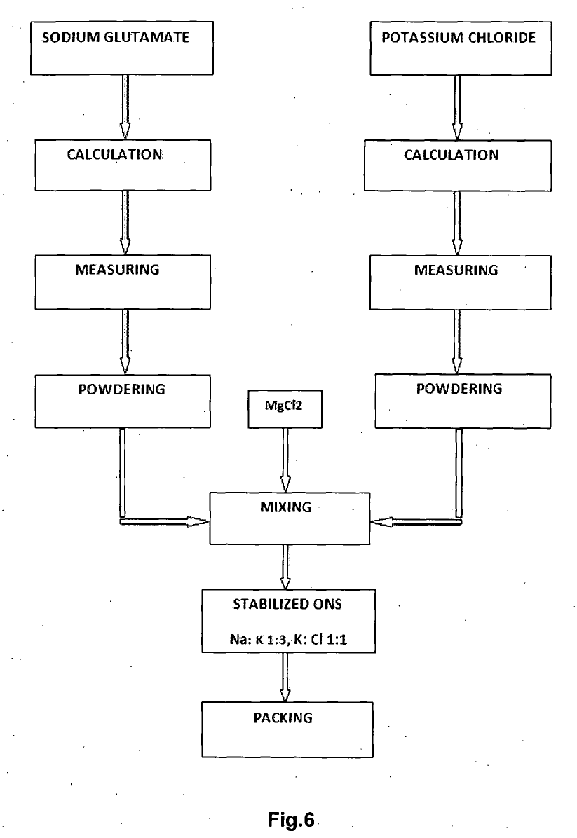

In another embodiment of the invention as depicted in FIG. 6 sodium to potassium ratio is 1:3 and potassium to chloride ratio is around 1:1. As per Daily Value minimum requirement of potassium per day is 3500 mg and chloride is 3400 mg per day. In this embodiment sodium glutamate is used as sodium source and potassium chloride as source of potassium and chloride and magnesium chloride as a source of chloride and as taste stabilizer. This optimized nutrient salt when used as cooking salt gives special umami taste (pleasant savoury taste) to food along with nutrients potassium, sodium, magnesium and chloride. Similarly sodium bicarbonate can be used as a source of sodium along with potassium chloride as source of potassium and chloride and calcium carbonates stabilizer. The resultant optimized nutrient salt composition when used as cooking salt as the additional advantages of leavening effect, acidity reduction and reduction in formation of calcium oxalate stones in the urinary tract due to formation of calcium oxalate crystals in the intestinal lumen and thereby preventing oxalate absorption in to the blood and calcium oxalate crystal formation in the urinary tract. In addition alkalisation of urine due to sodium bicarbonate will prevent uric acid crystal deposition in urinary tract.

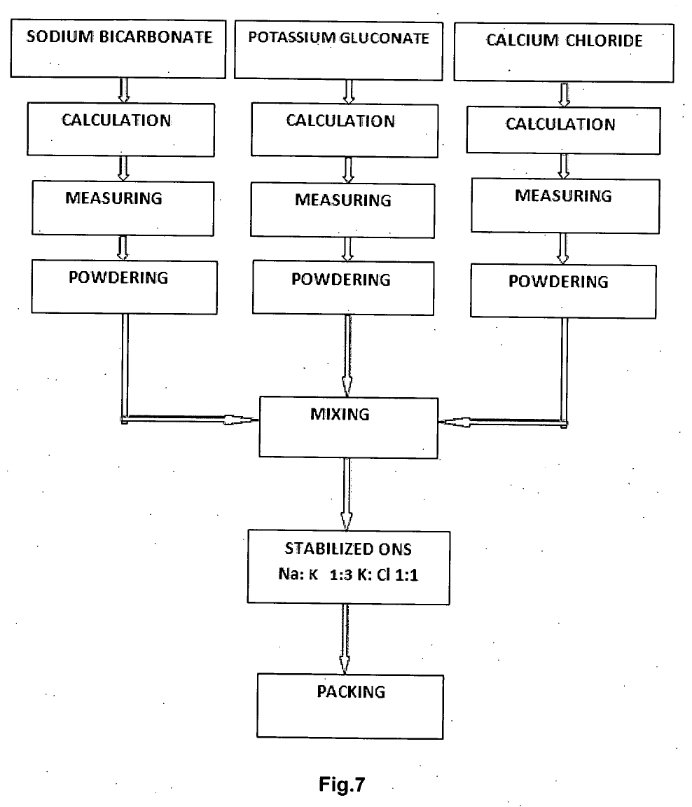

In another embodiment of the invention as depicted in FIG. 7 sodium bicarbonate is used as sodium source and potassium gluconate as source of potassium and calcium chloride as source of chloride and as taste stabilizer. This optimized nutrient salt when added in food gives essential nutrient calcium along with sodium, potassium and chloride. Due to the presence of bicarbonate ion, and is use full in treatment of acidic urine and prevention and treatment of uric acid stones in urinary tract. Presence of calcium ions in the food from calcium chloride will prevent absorption of oxalic acid in the food by forming calcium oxalate in the intestine and excreted in stool there by prevent oxalic acid absorption and formation of calcium oxalate crystals in urine.

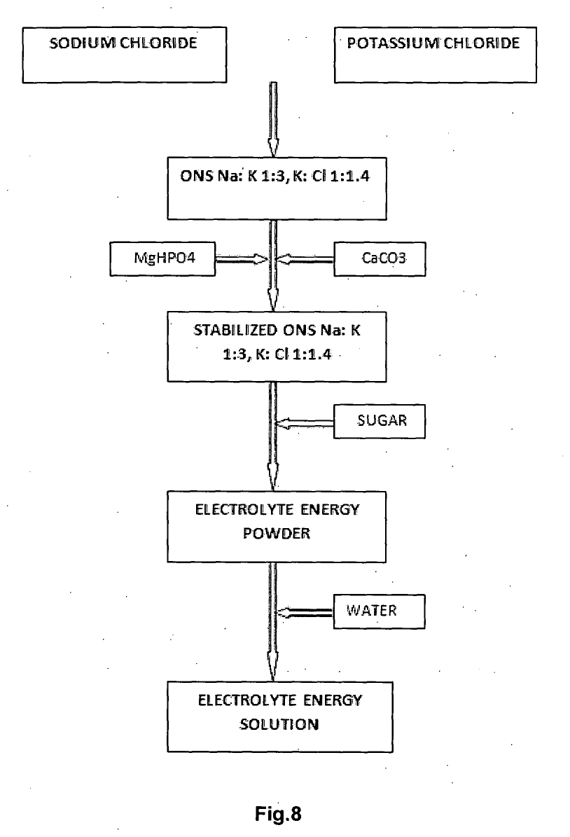

In another embodiment of the invention as depicted in flow diagram FIG. 8 shows the method of production of enriched optimized nutrient salt using sodium chloride, potassium chloride, calcium carbonate and sugar. This electrolyte energy powder which can be reconstituted with addition of water to electrolyte energy solution with elemental sodium to potassium ratio optimal using stabilized optimized nutrient salt and sugar. A composition comprising optimized nutrient salt and carbohydrate including polysaccharides, monosaccharides, disaccharides and oligosaccharides in 1:9 to 1:20 ratio makes electrolyte energy powder. The electrolyte energy solution/drink is prepared by adding 50 to 100 gm electrolyte energy powder dissolved with 1 litre water. This solution can provide water, major electrolytes Na, K and Cl in optimal ratio and glucose for energy which can be used as oral rehydration fluid. All our activities depends on integration and evaluation of our sensory input at brain and conscious and subconscious responses through skeletal, cardiac and smooth muscles in response to the sensory input. A continuous supply of electrolytes sodium, potassium and chloride and glucose for energy is essential for nerve conduction and sustaining life. Optimized nutrient salt with glucose, water and other nutrients optimizes the cellular activities and can be used as oral rehydration fluid or as intravenous fluid in medical conditions where fluid and electrolyte correction is needed.

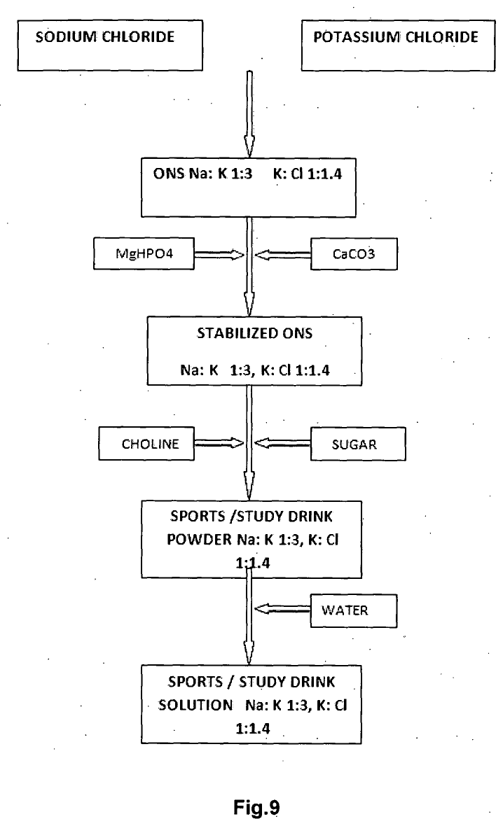

In another embodiment of the invention as depicted in flow diagram FIG. 9 shows the method of production of sports/study drink and sports/study drink solution with elemental sodium, potassium and chloride, ratio optimal using optimized nutrient salt stabilized with calcium carbonate and magnesium phosphate; sugar and a choline source including choline bitartarate, choline chloride, lecithin, phosphatidylcholine or any other choline source.

Choline is an essential water soluble vitamin which is needed for the synthesis of the neurotransmitter acetyl choline. Studies show choline deficiency in modern diet. During sports and intellectual activities choline is needed for acetylcholine production and continuous supply can boost performance. Another embodiment of the invention sports/study drink concentrate containing optimized nutrient salt 5-15% preferably 10%, choline bitartarate 1-8% preferably 5%, and carbohydrate includes glucose, sucrose, fructose, oligosaccharide, polysaccharide, or any other form of edible carbohydrate 80-95% preferably 85%. Enriched optimized nutrient salt containing 10% optimized nutrient salt, 5% choline bitartarate and 85% sugar when reconstituted with water as 5% solution give almost similar electrolyte composition and taste of tender coconut water with extra benefit of choline. If taken during physical activity or mental activity can improve performance. One liter of this sports/study drink solution contains around 1000 mg of choline, 1800 mg potassium, 2600 mg chloride, 600 mg sodium and 42.5 gm sugar. As this solution contains water, energy source as carbohydrate, electrolytes sodium, potassium and chloride in optimal proportion and choline for the synthesis of neurotransmitter acetylcholine, can be used as performance booster during physical and mental activities. It can also be used for the treatment of fatty liver and alcoholic liver diseases to supplement choline.

A further improvement of this embodiment of the invention was addition of pantothenic acid (vitamin B5) and vitamin C to further boost performance and endurance. Other electrolytes like calcium, magnesium, phosphate and sulfate, vitamins and amino acids may also be added to ensure continuous supply of these nutrients without exhausting the body stores in more demanding sports activity like marathon.

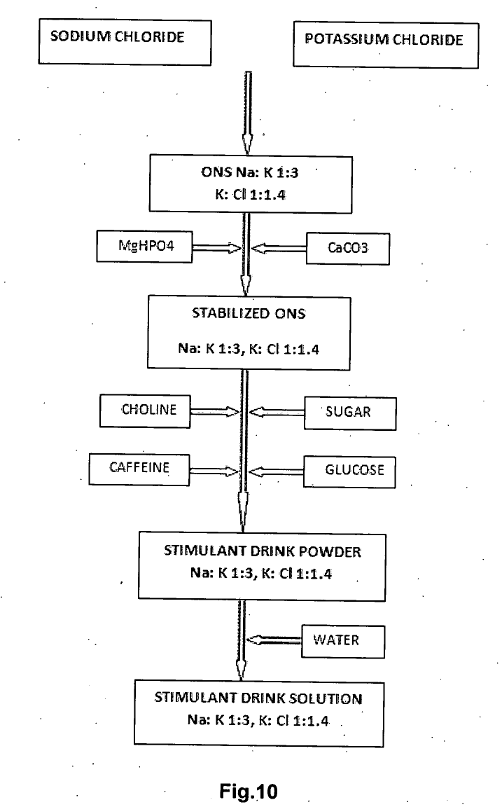

In another embodiment of the invention as depicted in flow diagram FIG. 10 shows the method of production of stimulant sports/study drink concentrate with elemental chloride, sodium and potassium in optimal ratio using stabilized optimized nutrient salt stabilized with calcium carbonate and magnesium phosphate, sugar, glucose, choline and caffeine. Other nutrients including minerals, electrolytes, vitamins, amino acids, fats/oils, fatty acids, proteins, stimulant drugs or stimulant herbal extracts also can give boosting effect. In an embodiment of the invention stimulant drink concentrate containing optimized nutrient salt 2-10% preferably 5%, choline tartrate 0.5-4% preferably 2.5%, caffeine 0.1-1% preferably 0.5%, calcium carbonate 0.1-1% preferably 0.5%, magnesium phosphate 0.1-1% preferably 0.5%, sodium ascorbate 0.05-2% preferably 0.1%, calcium pantothenate 0.01-0.2% and carbohydrate selected from a group glucose, sucrose, fructose, oligosaccharide, polysaccharide or a combination thereof 85-95% preferably 91%. 5 to 15% solution of this enriched optimized salt solution during physical activity or mental activity can improve performance and endurance and can be effectively used as stimulant sports/study drink.

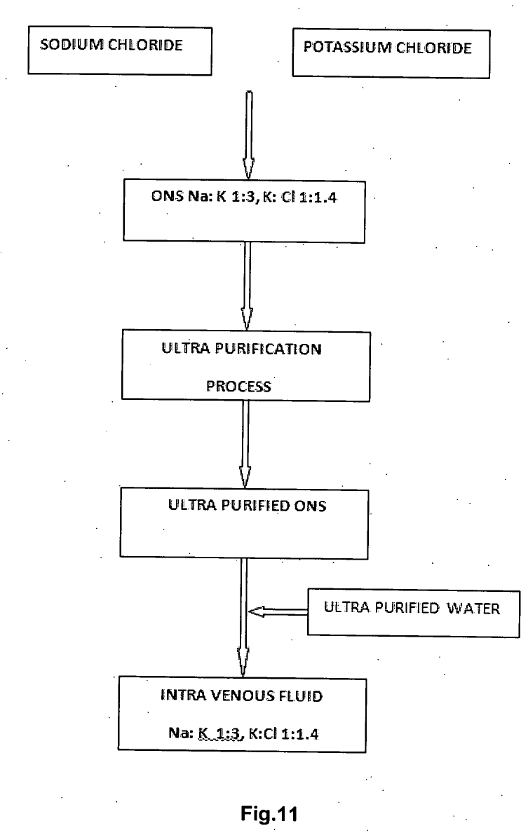

In another embodiment of the invention as depicted in flow diagram FIG. 11 shows the method of production of intravenous fluid with optimal elemental chloride, sodium and potassium ratio using optimized nutrient salt. Method of production involves dissolving ultra-purified optimized nutrient salt in ultra-purified water as per standard pharmaceutical technologies.

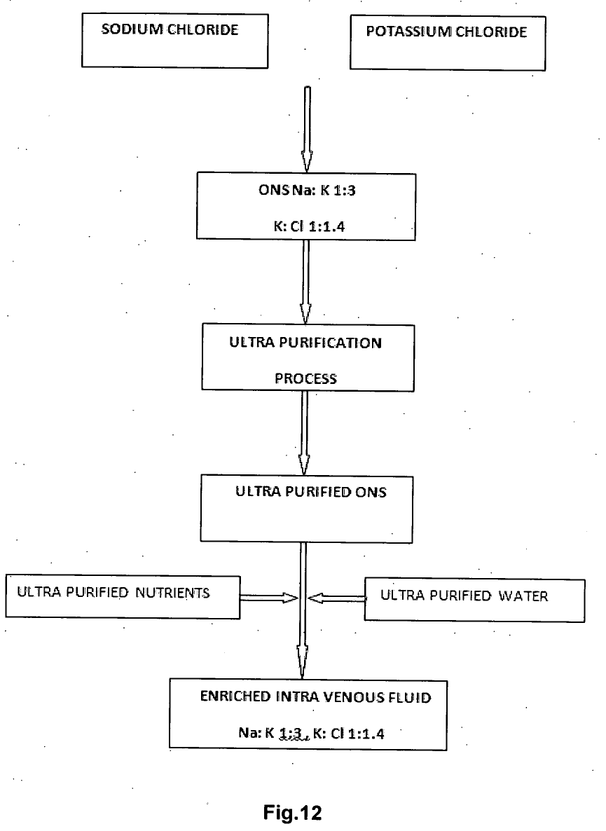

In another embodiment of the invention as depicted in flow diagram FIG. 12 shows the method of production of intravenous nutrition fluid using optimized nutrient salt. Method includes enriching the optimized nutrient salt for production of intravenous fluid, or optimized nutrient salt containing intra venous fluid with glucose, vitamins, amino acids, minerals, triglycerides, fatty acids and, or a combination thereof as per standard pharmaceutical procedures.

The constituents of salts in this optimized nutrient salt composition and, or its solution when taken as food/food supplement/nutrition acts as means for stabilization of sodium potassium pump at cellular level in multicellular organisms. Optimized nutrient salt composition is less salty than common salt and can provide enough electrolytes potassium, chloride and sodium in calorie restricted diet of obese patients and easily compensate for electrolyte loss through sweat, stool, plasma, serum or urine and can be used as oral rehydration fluid or intravenous fluid. It can also function as cooking salt, table salt, preservative for food and food ingredient.

### EXAMPLES

### Production

Food grade sodium chloride, sodium bicarbonate, sodium glutamate, potassium chloride, potassium gluconate, calcium chloride and magnesium chloride was used for the production of optimized nutrient salt. For stabilization of the salt composition food grade calcium carbonate, calcium chloride, magnesium carbonate, and magnesium phosphate were used. For enrichment with nutrients sugar, glucose, choline bitartarate, sodium ascorbate, calcium pantothenate, caffeine and water were used.

### Example 1

7 kg powdered potassium chloride and 3 kg of powdered sodium chloride were mixed in a mixing machine for 10 minutes at 500 RPM. 200 gm of powdered calcium carbonate was added into the salt composition as stabilizer. Again thorough mixing was done for 10 minutes at 500 RPM to get an even mix. The resultant stabilized optimized nutrient salt was stored in air tight containers to avoid contact with moisture. In this composition sodium to potassium ratio is around 1:3 and potassium to chloride ratio is around 1:1.4.

### Example 2

700 gm powdered potassium chloride and 300 gm of powdered sodium chloride were mixed in a mixing machine for 10 minutes at 500 RPM. 20 gm of powdered calcium carbonate was added in to the salt composition as stabilizer. Again thorough mixing was done for 10 minutes at 500 RPM to get an even mix. 9 kg powdered sugar was added into the stabilized optimized nutrient salt composition. Again thorough mixing was done for 10 minutes at 500 RPM to get an even mix. The resultant electrolyte energy powder containing optimized nutrient salt was stored in air tight containers to avoid contact with moisture. The electrolyte energy powder when reconstituted with water as 5% to 15% electrolyte energy solution was suitable for use as oral rehydration fluid.

### Example 3

700 gm powdered potassium chloride and 300 gm of powdered sodium chloride were mixed in a mixing machine for 10 minutes at 500 RPM. 10 gm of powdered calcium carbonate and 10 gm magnesium phosphate was added in to the salt composition as stabilizer. Again thorough mixing was done for 10 minutes at 500 RPM to get an even mix. 250 gm of powdered choline bitartarate was added into the stabilized optimized nutrient salt composition and thorough mixing was done for 10 minutes at 500 RPM to get an even mix. 9 kg powdered sucrose was added into the enriched optimized nutrient salt composition. Again thorough mixing was done for 10 minutes at 500 RPM to get an even mix. The resultant sports/study drink concentrate containing enriched optimized nutrient salt was stored in air tight containers to avoid contact with moisture.

### Example 4

700 gm powdered potassium chloride and 300 gm of powdered sodium chloride were mixed in a mixing machine for 10 minutes at 500 RPM. 20 gm of powdered calcium carbonate and 10 gm magnesium phosphate was added in to the salt composition. Again thorough mixing was done for 10 minutes at 500 RPM to get an even mix. 250 gm of powdered choline bitartarate and 20 gm of sodium ascorbate and 2 gm calcium pantothenate were added into the stabilized optimized nutrient salt composition and thorough mixing was done for 10 minutes at 500 RPM to get an even mix. 9 kg powdered sucrose and 5 kg powdered glucose was added into the enriched optimized nutrient salt composition. Again thorough mixing was done for 10 minutes at 500 RPM to get an even mix. The resultant sports/study drink concentrate containing optimized nutrient salt enriched with carbohydrate sucrose and glucose, vitamin c, choline and calcium pantothenate (vitamin B5) was stored in air tight containers to avoid contact with moisture.

### Example 5

700 gm powdered potassium chloride and 300 gm of powdered sodium chloride were mixed in a mixing machine for 10 minutes at 500 RPM. 10 gm of powdered calcium carbonate and 10 gm magnesium phosphate dibasic was added in to the salt composition. Again thorough mixing was done for 10 minutes at 500 RPM to get an even mix. 250 gm of powdered choline bitartarate, 2 gm calcium pantothenate and 40 gm caffeine were added into the stabilized optimized nutrient salt composition and thorough mixing was done for 10 minutes at 500 RPM to get an even mix. 12 kg powdered sucrose were added into the enriched optimized nutrient salt composition. Again thorough mixing was done for 10 minutes at 500 RPM to get an even mix. The resultant stimulant sports/study drink concentrate containing optimized nutrient salt enriched with sucrose, choline, calcium pantothenate, calcium, magnesium and phosphorus was stored in air tight containers to avoid contact with moisture.

### Example 6

3.5 kg powdered potassium chloride and 5 kg of powdered sodium glutamate were mixed in a mixing machine for 10 minutes at 500 RPM. 200 gm of powdered magnesium carbonate was added in to the salt composition as stabilizer. Again thorough mixing was done for 10 minutes at 500 RPM to get an even mix. The resultant stabilized optimized nutrient salt was stored in air tight containers to avoid contact with moisture. In this composition sodium to potassium ratio is around 1:3 and potassium to chloride ratio is around 1:1.

### Example 7

7 kg powdered potassium chloride, 6.5 kg of powdered sodium bicarbonate and were mixed in a mixing machine for 10 minutes at 500 RPM. 200 gm of powdered magnesium chloride was added in to the salt composition as stabilizer. Again thorough mixing was done for 10 minutes at 500 RPM to get an even mix. The resultant stabilized optimized nutrient salt was stored in air tight containers to avoid contact with moisture. In this composition sodium to potassium ratio is around 1:3 and potassium to chloride ratio is around 1:1.

### Example 8

10.5 kg powdered potassium gluconate and 3.25 kg of powdered sodium bicarbonate and 2.15 kg calcium chloride were mixed in a mixing machine for 10 minutes at 500 RPM to get an even mix. The resultant stabilized optimized nutrient salt was stored in air tight containers to avoid contact with moisture. In this salt composition sodium to potassium ratio is around 1:3 and potassium to chloride ratio is around 1:1.

### Example 9

700 gm pharma grade ultra-purified powdered potassium chloride and 300 gm pharma grade ultra-purified powdered sodium chloride were mixed in a mixing machine for 10 minutes at 500 RPM. 9 kg pharma grade ultra-purified powdered glucose was added into the stabilized optimized nutrient salt composition. Again thorough mixing was done for 10 minutes at 500 RPM to get an even mix. The resultant electrolyte energy powder containing optimized nutrient salt was stored in air tight containers to avoid contact with moisture.

### Example 10

Intravenous fluid was prepared using enriched optimized nutrient salt prepared by a process given in example 9.1 kg ultra purified optimized nutrient salt was dissolved in 20 liter ultra-purified water for injection using sterile pyrogen free techniques as per standard pharmaceutical procedures. This 5% enriched optimized nutrient salt solution contains 4.5% glucose, 0.35% potassium chloride and 0.15% sodium chloride.

### Case Studies

The optimized nutrient salt compositions and the nutrient enriched compositions produced as the embodiments of the present invention by the said method as described in Example 1 to 10 were administered to those who are needy and found to give encouraging results. Few of the relevant case studies are provided in the following examples which fully justify our contention that the surprising findings are attributable to synergetic effect of the elements in special proportion in the composition, involved in the state of the art optimized nutrient salt composition according to the present invention.

### Example 11

20 patients of age 30-60 years with mild to moderate hypertension-systolic BP 140 to 180 mm of mercury and diastolic BP 90 to 110 mm of mercury were taken for study. Patients were divided into two groups. Both the groups were evaluated with routine urine test, blood test including hematology and biochemistry. Patients in both groups were advised to follow DASH diet. The first group was advised to reduce common salt less than 3 gm per day. The second group was asked to take up to 10 gm optimized nutrient salt prepared by a process as given in example 1 as cooking/table salt per day replacing common salt they normally use. Patients in both the groups were asked to keep record of blood pressure and diet for a period of one month.

At the end of 1 month, patients were evaluated on ability to stick to DASH diet and blood pressure control by monitoring blood pressure chart of the month, current blood pressure and diet record of the month. All the patients in the second group on optimized nutrient salt showed substantial reduction in blood pressure by 10 to 20 mm of mercury in systolic pressure and 6 to 12 mm of mercury in diastolic pressure and the second group of people were able to stick to recommended level of sodium intake per day below 1500 mg and potassium intake above 4500 mg without difficulty as they were taking 5 to 10 gm optimized nutrient salt instead of common salt. The patients in first group who were asked to follow dash diet without optimized nutrient salt were not able to reach the target reduction in sodium and increase in potassium as prescribed by DASH diet and reduction in blood pressure was less significant. At the end of one month reduction in systolic blood pressure was around 4 to 8 mm mercury and diastolic pressure 2 to 4 mm of mercury which was significantly lower than the second optimized nutrient salt using group.

10 gm optimized nutrient salt of example 1 contains 1200 mg sodium, 3600 mg potassium and 5200 mg chloride. If the patient takes 10 gm optimized nutrient salt the sodium intake is only 1200 mg and to reach target potassium only extra 900 mg is needed and can be achieved from regular food. If the patient takes 10 gm common salt the sodium intake will be around 4000 mg that is very high, even 5 gm common salt contains around 2000 mg sodium and exceeds sodium intake above permissible limit 1500 mg as per DASH diet. To meet recommended dietary intake of chloride one has to take at least 6 gram of common salt because only very few food sources contain substantial amount of chloride and we depend on common salt for chloride. Reduced amount of chloride in the diet leads to tiredness and fatigue resulting in reduction in productivity. If one reduces the common salt intake to 3 gm, the chloride intake may go down below 2000 mg and which will have negative impact on health. Use of optimized nutrient salt instead of common salt makes the DASH diet prescription easy to follow and practical there by control hypertension easily and ensure all major electrolytes intake optimal and as per latest scientific recommendations.

When optimized nutrient salt is given as table salt and as cooking salt it was easy to meet the recommendation. Saltiness of the optimized nutrient salt is around 50% and sodium content is 30% compared to common salt. So it is easy to add around 10 gm of optimized nutrient salt during food preparation. The second group who were given optimized nutrient salt supplement was able to meet the target of daily intake of chloride, sodium and potassium easily and thereby achieve control of hypertension without difficulty.

### Example 12

20 patients of age 18 to 60 years with overweight BMI above 25 were taken for study. Patients were divided into two groups. Both the groups were evaluated with routine urine test, blood test including hematology, thyroid function and biochemistry. Patients in both groups were advised to reduce calorie in the diet to around 1000 calories per day and exercise for one hour every day. Both the groups were given vitamins and mineral supplement tablets. The second group was asked to drink enriched optimized salt solution containing 50 gm enriched optimized nutrient salt prepared by a process as given in example 2, dissolved in 1000 ml water during the active hours of the day especially during exercise. Patients in both the groups were asked to keep daily diet and calorie intake chart, exercise chart and weight chart for a period of one month.

At the end of 1 month patients were evaluated on ability to stick to low calorie diet and exercise and extent of weight reduction by evaluating daily diet and calorie intake chart, exercise chart and weight chart. Patients in the first group were able to stick to low calorie diet and exercise in the first week with reduction in weight of 0.5 to 1 kg in the initial week, but they were not able to maintain the diet or exercise due to tiredness and fatigue and by the end of one month there was no significant weight reduction. All the patients in the second group on enriched optimized nutrient salt solution were able to stick to daily exercise and reduce the calorie intake as per recommendation and showed significant reduction in weight because they were given continuous supply of the major electrolytes with optimal sodium, potassium and chloride ratio and sugar as a source of continuous energy supply for nerve cells which prevents fatigue and kept the cells and body energetic. At the end of one -month the second group was able to reduce 2 to 4 kg weight which was statistically significant.

Major electrolytes at cellular level sodium, potassium and chloride are not stored in the body like calcium and magnesium. For successful maintenance of calorie restricted diet ensuring a steady supply of these macro minerals sodium, potassium and chloride in the diet is important. A steady supply of glucose is needed to meet the energy requirement of nerve cells. Enriched optimized nutrient salt solution containing sucrose ensures the proper functioning of sodium potassium pump at cellular level and steady supply of energy and electrolytes to nerve cells for message conduction which enabled the second group of patients to stick to low calorie diet and exercise without tiredness.

### Example 13

A team of football players practicing for competition were taken for study. On day one and three they were given 2000 ml of sugar water containing 90 gm sugar during the practice hours. On second and fourth day they were given 2000 ml enriched optimized nutrient salt solution containing 100 gm of enriched optimized nutrient salt prepared by a process as given in example 3. All the team members were asked to self-evaluate and record their performance on each day and the study manager observed the performance and interviewed each member and recorded the inference on all four study days. There was significant improvement on performance and endurance for all team members on day 2 and day 4 compared to day 1 and day 3.

During sports activity contraction of muscle and coordination of body movements require series of impulse transmissions in the nervous system and this electrical activity depends on movement of sodium and potassium ions across the nerve fibers by sodium potassium pump. The gradient of sodium and potassium concentration across the cell membrane is around 1:3. Ensuring the dietary sodium potassium ratio 1:3 with optimized nutrient salt solution on day 2 and 4 for the team members helped to optimize the impulse transmission and there by performance in sports. Lost sodium, potassium and chloride in sweat with exertion was replaced with continuous supply of optimized nutrient salt solution on day 2 and day 4. Continuous supply of electrolyte in optimal proportion along with choline, sugar and water enabled the team members to perform better on day 2 and day 4 compared to sugar and water on day 1 and day 3.

Acetylcholine is the neurotransmitter at neuromuscular junction and in peripheral nervous system and neuromodulator in central nervous system. Continuous supply of choline also has a significant role in better performance and endurance for the team on day 2 and day 4. Continuous excitation and relaxation of the nerve and muscle cells during a sports activity needs continuous supply of electrolytes and choline for optimal results.

### Example 14

10 students preparing for competitive examination was taken for study. On day one, two and three, they were given 1500 ml of sugar water with fresh lime juice containing 90 gm sugar during the study hours. On day four, five and six they were given 1500 ml stimulant optimized nutrient salt (enriched with caffeine, choline tartrate and sucrose) solution containing 100 gm of enriched optimized nutrient salt prepared by a process as given in example 5. All the study members were asked to self-evaluate and record their performance in terms of a) time spend for study, b) portions covered and c) recollection ability on each day and the study manager observed the performance and interviewed each member and recorded the inference on all six days. There was significantly better performance for all the ten students on all three aspects; time spend for study, portions covered and recollection ability on day four, five, and six while the students were on stimulant optimized nutrient study drink compared to day one, two and three while the students were on lime juice.

During study brain activity and impulse transmissions in the central nervous system are activated. This electrical activity depends on movement of sodium and potassium ions across the nerve fibers by sodium potassium pump and neurotransmitter acetylcholine at nerve junctions. The gradient of sodium and potassium concentration across the cell membrane is around 1:3. Ensuring continuous supply of electrolytes with sodium potassium ratio 1:3, energy from carbohydrate in the form of glucose, choline from choline bitartarate for the continuous supply of neurotransmitter acetylcholine and stimulant caffeine which act by improving the microcirculation in tissues and adenosine receptor blockage, from 1500 ml enriched optimized nutrient salt stimulant drink on day 4, 5 and 6 helped the study group to optimize the impulse transmission and brain cell function and there by performance in intellectual activity.

Acetylcholine is the neurotransmitter at neuromuscular junction and in peripheral nervous system and neuromodulator in central nervous system. Pantothenic acid is needed for the synthesis of acetyl CoA which is needed for the synthesis of acetylcholine along with choline. Continuous supply of electrolytes sodium, chloride, potassium, calcium, magnesium and phosphate along with choline, pantothenic acid, glucose and caffeine in stimulant study drink helps better performance in intellectual activity.

### Example 15

Two sets of 1000 gm tender mangoes were put in two separate sterile glass bottles. First bottle was filled with common salt solution up to the neck of the bottle and the second bottle was filled with optimized nutrient salt solution prepared by a process as given in example 1 of matching saltiness. Saltiness of optimized nutrient salt is only half of common salt. While the common salt solution used was 2.5% and the optimized nutrient salt solution was 5%. Bottle closed with cap and kept for seasoning for 45 days in room temperature around 25 to 35 degree centigrade.

On observation after 45 days the mangoes put in common salt solution showed fading of green color, surface was irregular and shrunken and there was a thin layer of fungal growth at the surface of the solution. The mangoes in the second bottle in optimized nutrient salt solution also showed fading in green color but the surface was smooth without shrinking and the surface of the solution was clear and there was no fungal growth.

This shows the optimized nutrient salt is a better preservative than the common salt because the preserved vegetable retained its texture and shape and there was no fungal growth, which means it is better preserved. Even though double the quantity of optimized nutrient salt was used for getting equal saltiness, the total sodium content was only around 60% compared to common salt solution. Because the sodium content is low and the potassium content in optimized nutrient salt solution is high, the intracellular fluid was not drawn out, therefore there was no shrinking of the vegetable. The preservative effect of common salt which inhibits growth of microorganism is due to the chloride content. Chloride ions in optimized nutrient salt solution was nearly double compared to the common salt solution with matching saltiness. While using optimized nutrient salt the preservative effect is almost doubled with a low sodium content of almost 60% and added nutritional benefit of potassium which is deficient in modern industrialized food.

### Example 16

Two sets of 1000 gm chicken pieces were put in two separate sterile glass containers. Chicken in first container was mixed with 25 gm common salt and chicken in second container was mixed with 50 gm of optimized nutrient salt prepared by a process as given in example 1 for matching saltiness. Saltiness of optimized nutrient salt is only half of common salt. Closed containers were kept in refrigerator at 2 to 6 degree centigrade for 10 days.

On observation after 10 days, in the first container chicken pieces were shrunken, reddish fluid oozed out from the chicken pieces and had slight putrid smell. But in the second container there was no fluid or putrid smell and chicken pieces looked fresh and edible in appearance and was edible and tasty after cooking.

This shows the optimized nutrient salt is a better preservative than the common salt because the preserved meat retained its consistency and freshness, indicating that it was better preserved. Even though double the quantity of optimized nutrient salt was used for getting equal saltiness, the total sodium content was only around 60% compared to common salt. Because the sodium content is low and the potassium content in optimized nutrient salt is high, the intracellular fluid was not drawn out, because of which there was no shrinking or fluid collection. The preservative effect of salt which inhibits growth of microorganism is due to the chloride content. Chloride ions in the optimized nutrient salt was nearly double compared to the common salt with matching saltiness. While using optimized nutrient salt the preservative effect is almost doubled with a low sodium content of almost 60% and added nutritional benefit of potassium which is deficient in modern industrialized food. While using common salt as preservative in meat some amount of potassium and other nutrients will be lost from the meat in the oozed out fluid which reduces the nutritional value of the meat. This shows optimized nutrient salt is significantly better preservative than common salt with additional nutritional benefit.

We have brought out the novel features of the invention by explaining some of the preferred embodiments under the invention, enabling those skilled in the art to understand and visualize our invention. Numerous changes can be made without departing from the concept discussed herein. Hence it is to be understood that within the scope of the claims provided herein, the invention may be practiced otherwise then as specifically described herein.

We claim:

1. An optimized nutrient salt composition capable of using as a food, a food ingredient, a food supplement, table salt, a preservative, a food additive, a seasoning agent, an ingredient for oral rehydration fluid, an ingredient for pharmaceutical preparation of intravenous fluids, an ingredient of beverages, energy drinks, sports/study drinks and stimulant drinks for humans, pets and livestock and for plant nutrition, comprising: elemental sodium and potassium in said composition in the ratio ranging from 1:1.5 to 1:7 and elemental potassium to chloride in the ratio ranging from 1:1 to 1:1.5 in the composition.

2. The optimized nutrient salt composition as claimed in claim 1, comprising: calcium and, or magnesium salt as taste stabilizer.

3. The optimized nutrient salt composition as claimed in claim 1, wherein: ratio of elemental sodium to potassium is 1:3 and elemental potassium to chloride is 1:1.4.

4. The optimized nutrient salt composition as claimed in claim 1, wherein: ratio of elemental sodium to potassium is 1:3 and ratio of elemental potassium to chloride is 1:1.

5. The optimized nutrient salt composition as claimed in claim 1, wherein: a) source for sodium is selected from sodium chloride, sodium glutamate, sodium bicarbonate, sodium gluconate, sodium citrate, sodium phosphate, sodium ascorbate, sodium iodide, any other edible sodium salts or a combination thereof; b) source for potassium is selected from potassium chloride, potassium phosphate, potassium bicarbonate, potassium gluconate, potassium aspartate, potassium orotate, potassium glutamate, potassium citrate, potassium iodide, any other edible potassium salts or a combination thereof; and c) source for chloride is selected from sodium chloride, potassium chloride, calcium chloride, magnesium chloride, zinc chloride, choline chloride, any other edible chloride salts or a combination thereof.

6. The optimized nutrient salt composition as claimed in claim 1, wherein: taste stabilizing calcium and, or magnesium salts are selected from a group comprising of calcium carbonate, magnesium carbonate, magnesium oxide, calcium phosphate, magnesium phosphate, calcium citrate, calcium chloride, magnesium chloride, any other edible calcium or magnesium salt or a combination thereof.

7. The optimized nutrient salt composition as claimed in claim 1, wherein: said composition is enriched with a) one or more nutrients selected from a group comprising of vitamins, minerals, amino acids, proteins, fats/oil, fatty acids, carbohydrates, water, any other nutrients or a combination thereof; b) one or more food additives selected from a group comprising of anti-caking agents, crystal modifying agents, flavoring agents, flavor modifying agents, coloring agents, preservatives, any other food additives or a combination thereof.

8. A method of preparation of the optimized nutrient salt composition, comprising:
a) selecting the source of salts from edible salts of food grade/pharmaceutical grade salts of sodium, salts of potassium and chloride salts;
b) calculating the amount of chloride, sodium and potassium in the selected salts considering the chemical formula of the salt selected;
c) finding out the proportion of each salt to be taken to maintain the ratio of sodium to potassium in the salt composition in the range of 1:1.5 to 1:7 and potassium to chloride in the range of 1:1 to 1:1.5 using standard chemistry calculation methods;
d) measuring the selected salts as per calculation;
e) powdering the salts in a pulverizer;
f) combining the salt composition in a mixing chamber at 200-500 RPM for 10 to 30 minutes to get an even composition;
g) taste stabilization by adding calcium and, or magnesium salt in to the combination;
h) mixing the composition in a mixing chamber at 200-500 RPM for 10 to 30 minutes to get an even composition;
i) enriching with nutrients selected from vitamins, minerals, amino acids, proteins, fats/oil, fatty acids, carbohydrates, or any other nutrients; and food additives selected from anti caking agents, crystal modifying agents', flavoring agents or any other food additives; and
j) packing the product of step i) in to air tight containers to avoid moisture ingress.

9. The method of preparation of the optimized nutrient salt composition as claimed in claim 8, wherein: edible calcium salt and, or magnesium salt is added in to the composition as taste stabilizer and the quantity added is 0.5 to 5% of the composition.

10. The method of preparation of the optimized nutrient salt composition as claimed in claim 8, wherein: the said composition is enriched with nutrients selected from a group comprising of vitamins, minerals, amino acids, proteins, fats/oils, fatty acids, carbohydrates or a combination thereof.

11. The method of preparation of optimized nutrient salt composition as claimed in claim 8, wherein: stabilized optimized nutrient salt is enriched with a carbohydrate selected from a group comprising monosaccharide, disaccharide, oligosaccharide and polysaccharide or a combination thereof to produce energy electrolyte powder.

12. The method of preparation of optimized nutrient salt composition as claimed in claim 8, wherein: stabilized optimized nutrient salt is enriched with a) a choline source selected from a group comprising of choline bitartarate, choline chloride, lecithin, phosphatidylcholine and any other choline source or a combination thereof; and b) a carbohydrate selected from a group comprising monosaccharide, disaccharide, oligosaccharide and polysaccharide or a combination thereof to produce sports/study drink powder.

13. The method of preparation of optimized nutrient salt composition as claimed in claim 1, wherein: stabilized optimized nutrient salt is enriched with a) a stimulant selected from a group comprising of caffeine, any stimulant drug, coffee extract, tea extract, coco extract any other edible stimulant herbal extract or combination thereof; b) choline source selected from a group comprising of choline bitartarate, choline chloride, lecithin, phosphatidylcholine, any other edible choline source or combination thereof; c) a carbohydrate selected from a group comprising monosaccharide, disaccharide, oligosaccharide and polysaccharide or a combination thereof to produce stimulant sports/study drink powder.

14. The method of preparation of the optimized nutrient salt composition as claimed in claim 8, wherein:
a) optimized nutrient salt composition is subjected to filtration and ultra-purification before mixing with ultra-purified water to make intra venous fluid as per known pharmaceutical technologies; and
b) the product of step a) is enriched with one or more ultra-purified nutrients selected from a group comprising of glucose, vitamins, minerals, amino acids, proteins, triglycerides, fatty acids, any other parenteral nutrients or a combination thereof to make it as intravenous nutrient fluid for parenteral nutrition.

15. The optimized nutrient salt composition as claimed in any of the preceding claims, wherein; said composition is a means for a) stabilization of sodium potassium pump at cellular level in multicellular organisms; b) providing electrolytes in calorie restricted diet of obese patients; and c) compensating electrolyte loss through sweat, stool, plasma, serum and urine.
